(19) United States
(12) Patent Application Publication
(10) Pub. No.: US 2014/0016179 A1
(43) Pub. Date: Jan. 16, 2014

(54) PIGMENT-BASED INKS

(71) Applicants: Zhang-Lin Zhou, Palo Alto, CA (US); Qin Liu, Corvallis, OR (US)

(72) Inventors: Zhang-Lin Zhou, Palo Alto, CA (US); Qin Liu, Corvallis, OR (US)

(21) Appl. No.: 13/547,832

(22) Filed: Jul. 12, 2012

(51) Int. Cl. C07D 407/14; C09D 11/00; G02F 1/167; C07D 407/12

(52) U.S. Cl. 359/296; 549/551; 549/554; 549/556; 549/555; 106/31.6; 106/31.75; 106/31.88; 106/31.85; 106/31.86; 106/31.65

(57) ABSTRACT

A compound is disclosed. The compound has a general structure:

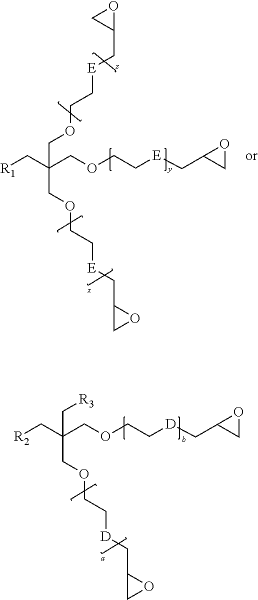

wherein R₁, R₂, and R₃ are each independently selected from the group consisting of hydrogen, substituted saturated hydrocarbons, non-substituted saturated hydrocarbons, substituted unsaturated hydrocarbons, and non-substituted unsaturated hydrocarbons; wherein E and D are each independently selected from the group consisting of CH₂, O, S, and NH; and wherein a, b, x, y, and z are each independently any whole number between 0 and 45, inclusive, wherein the sum of a and b is less than 45 or equal to 45 and wherein the sum of x, y, and z is less than 45 or equal to 45.

### BACKGROUND

Ultrathin, flexible, reflective electronic displays that look like print on paper are of great interest as they have potential applications in wearable computer screens, electronic paper, smart identity cards, and electronic signage. Electro-optical display technology, such as electrophoretic or electrokinetic display technology, is an important approach to this type of display medium. In electrophoretic or electrokinetic displays, pixel or segment electrodes, electrodes within the viewing area of a display that are electrically isolated, may control the local position of charged colorant particles in the ink by application of electric fields. The local position of the particles may influence the reflectance of such pixel or segment electrodes. Without subscribing to any particular theory, in electronic inks, particles that exhibit good dispersibility and charge properties in non-polar dispersing media may increase the stability of the ink and may improve the switching behavior of the ink, as further discussed below, which may increase the useful lifetime of the ink. Additionally, use of non-polar dispersing media in the electrophoretic or electrokinetic devices may minimize current leakage.

### BRIEF DESCRIPTION OF THE DRAWINGS

The detailed description will make reference to the following drawings, in which like reference numerals may correspond to similar, though perhaps not identical, components. For the sake of brevity, reference numerals having a previously described function may or may not be described in connection with other drawings in which they appear.

FIG. 1 depicts a cross-sectional view of one example of a stacked electro-optical display including an ink with the epoxide-based small molecular additive disclosed herein.

FIG. 2 is a schematic diagram of an example reaction scheme for how an epoxide-based small molecular additive may be grafted onto the surface of a pigment particle.

FIG. 3 is a schematic diagram of a specific example reaction scheme for how an epoxide-based small molecular additive may be grafted onto the surface of a phosphoric acid surface modified, silica coated pigment particle.

FIG. 4 is a schematic diagram of a specific example reaction scheme for how an epoxide-based small molecular additive may be grafted onto a hydroxyl group surface modified, silica coated pigment particle.

FIG. 5 is a schematic diagram of a specific example reaction scheme for how an epoxide-based small molecular additive may be grafted onto an amino group surface modified, silica coated pigment particle.

### DETAILED DESCRIPTION

Reference is now made in detail to specific examples of the epoxide-based small molecular additive and specific examples of inks including such additives. When applicable, alternative examples are also briefly described.

It is noted that, as used in this specification and the appended claims, the singular forms “a,” “an,” and “the” include plural referents unless the context clearly dictates otherwise.

As used herein, the “carrier fluid” is a fluid or medium that fills up a viewing area defined in an electronic ink display and is generally configured as a vehicle to carry pigment/colorant particles therein.

In the past, Hewlett-Packard has conducted research on displays utilizing electrokinetic/electrophoretic architecture that rely on pigment compaction, which permits both a colored state when the pigment particles are spread out and a clear state when the particles are tightly compacted within a cell or pixel, and wherein the repeated motion of spreading out and compacting is known as “switching”. (See e.g., Yeo, J. et al., “Electro-optical Display”, U.S. Pat. No. 8,018,642).

A bi-state display cell having a dark state and a clear state may be achieved using an electronic ink with charged pigment particles in an optically transparent fluid. A clear state may be achieved when the pigment particles are compacted, and a colored state may be achieved when the pigment particles are spread. For example, an electronic ink with charged white particles in a colored fluid may enable white and spot-color states, with the color of the colored state depending on the color of the fluid. The ink fluid may be colored by a dye, nanoparticles, pigments or other suitable materials. A white state may be achieved when the white particles are spread, and a colored state may be achieved when the white particles are compacted. By combining the white particles in the colored fluid with a colored resin on the back of the display cell, a tri-state display cell may be achieved.

An electrokinetic/electrophoretic display cell may use a three-dimensional architecture to provide a clear optical state. In this architecture, the geometrical shape of the display cell has narrowing portions in which electrokinetically/electrophoretically translated pigment particles may compact in response to appropriate bias conditions applied to driving electrodes on opposite sides of the display cell. The three-dimensional structure of the display cell may introduce additional control of electrokinetically/electrophoretically moving pigment particles. As a result, desired functionalities may be achieved with a developed and more stable electrokinetic/electrophoretic ink. In some examples, the driving electrodes may be passivated with a dielectric layer, thus eliminating the possibility of electrochemical interactions through the driving electrodes from direct contact with the electrokinetic/electrophoretic ink. In other examples, the driving electrodes may not be passivated, thus allowing electrochemical interactions with the electrokinetic/electrophoretic ink.

However, inks currently used in prior art displays may not work in stacked versions of an electrokinetic/electrophoretic architecture as such inks may be unable to achieve the level of compaction necessary to provide the clear states used in displays with stacked color architectures, as further described below.

For example, current commercial displays, such as displays manufactured by prior art display technology companies, utilize front to rear particle motion, which may only be able to provide opaque color and white states or black and white states. Additionally, such displays may not be capable of producing the clear states that allow displays to be used in stacked architectures, as further described below, and may rely on color filters to achieve full color. However, color filters, such as red, green or blue filters, may often be arranged side-by-side in a pixel, which may result in a decreased surface area within the pixel for modulating light and a decreased surface area within the pixel for reflecting incident light when not all of the color filters are required to produce a color. Accordingly, the resulting displayed image using color filters may have dull colors.

The ability to achieve a clear state, on the other hand, may allow displays to sit in a stacked architecture, and may allow the entire viewable area in the display to be used (i.e. the entire pixel of every pixel) when modulating light and reflecting incident light. The result may be a display able to achieve brighter colors and a better clear state. Additionally, because the entire viewable area in the display may be used to modulate light, such displays may also have a larger color gamut volume.

While progress toward developing working electronic inks for this stacked architecture has been made in the last few years, researchers continue to seek ways for improving the quality and versatility of these inks. (See e.g., Zhou, Z. L. et al., “Electronic Inks” published on Apr. 21, 2011 as WO2011/046562; Zhou, Z. L. et al., “Dual Color Electronically Addressable Ink” published on Apr. 21, 2011 as WO2011/046564; and Zhou, Z. L. et al., “Electronic Inks” published on Apr. 21, 2011 as WO2011/046563.)

In accordance with the teachings herein, an additive for inks is provided, wherein the additive is a reactive, functionalized, and sterically hindered small molecule based on functional epoxides. As used herein, a “small” molecular additive is a molecular additive that has a molecular weight of approximately 2000 or less. As used in this specification and the appended claims, “approximately” means having an upper bound of 10% above a recited value wherein the difference is due to formulations within a molecule (e.g. x, y, z, a, and b in structures (1) and (2) below). The small epoxide-based molecular additive may be grafted onto the surface of a pigment particle by a covalent bond reaction between a functional group on the pigment particle (e.g. hydroxyl group, amine group, carboxylic acid group, etc.) and the epoxide group on the small molecular additive. Finally, in some examples, the bonded small epoxide-based molecular additive and pigment particle may be added to an ink.

In electronic inks, the addition of the sterically hindered epoxide-based molecular additive described above may result in an electronic ink having more hydrophobic surfaces, which in turn, may improve the dispersibility and stability of pigment particles in the carrier fluid, which in turn, may result in electronic inks with improved lifetimes. Additionally, formation of a covalent bond between the functional group on the pigment particle and the epoxide group on the small molecular additive may increase steric stabilization, which may improve the stability of the ink. Finally, the increased steric stabilization may improve the color saturation properties and high switching speeds of the ink.

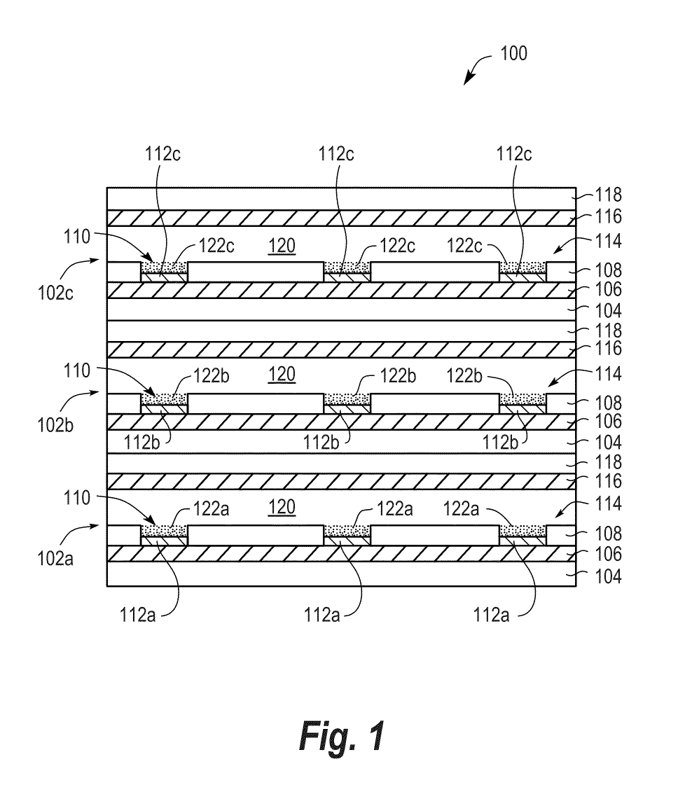

FIG. 1 illustrates a cross-sectional view of one example of a stacked electro-optical display 100 including an ink, such as an ink including the epoxide-based small molecular additive described herein. The electro-optical display 100 includes a first display element 102a, a second display element 102b, and a third display element 102c. The third display element 102c is stacked on the second display element 102b, and the second display element 102b is stacked on the first display element 102a.

In some examples, each display unit includes a first substrate 104, a first electrode 106, a dielectric layer 108 including reservoir or recess regions 110, thin layers 112, a display cell 114, a second electrode 116, and a second substrate 118. In other examples, the display unit does not include thin layers 112. The display cell 114 may be filled with the electronic ink 120, 122 disclosed herein including a carrier fluid 120 with pigment/colorant particles bonded to epoxide-based small molecular additives as described herein 122. In some examples, wherein thin layers 112 are included, the thin layers 112 may be opaque. In other examples, the thin layers 112 may be transparent. In examples wherein thin layers 112 are included, the thin layers 112 may include dielectric materials or conductive materials. In one specific example, a metallic material, such as nickel, may be used.

In examples wherein thin layers 112 are included, the first display element 102a includes thin layers 112a self-aligned within the recess regions 110. The first display element 102a also includes pigment particles 122a having a first color (e.g., cyan) for a full color electro-optical display. The second display element 102b includes thin layers 112b self-aligned within the recess regions 110. The second display element 102b also includes pigment particles 122b having a second color (e.g., magenta) for a full color electro-optical display. The third display element 102c includes thin layers 112c self-aligned within the recess regions 110. The third display element 102c also includes pigment particles 122c having a third color (e.g., yellow) for a full color electro-optical display. In other examples, the pigment particles 122a, 122b, and 122c may include other suitable colors for providing an additive or subtractive full color electro-optical display.

In the example illustrated in FIG. 1, in the electro-optical display 100 including the ink 120, 122, the first display element 102a, the second display element 102b, and the third display element 102c are aligned with each other. As such, the thin layers 112a, 112b, and 112c are also aligned with each other. In this example, since the recess regions 110 and the self-aligned thin layers 112a, 112b, and 112c of each display element 102a, 102b, and 102c, respectively, are aligned, the clear aperture for the stacked electro-optical display 100 may be improved as compared to a stacked electro-optical display without such alignment.

In an alternate example (not shown), the first display element 102a, the second display element 102b, and the third display element 102c may be offset from each other. As such, the thin layers 112a, 112b, and 112c are also offset from each other. In this example, since the recess regions 110 and the self-aligned thin layers 112a, 112b, and 112c are just a fraction of the total area of each display element 102a, 102b, and 102c, respectively, the clear aperture for the stacked electro-optical display 100 may remain high regardless of the alignment between the display elements 102a, 102b, and 102c. As such, the process for fabricating the stacked electro-optical display 100 may be simplified. The self-aligned thin layers 112a, 112b, and 112c may prevent tinting of each display element due to the pigment particles 122a, 122b, and 122c, respectively, in the clear optical state. Therefore, a stacked full color electro-optical display having a bright, neutral clear state and precise color control may be provided.

Turning now to the epoxide-based small molecular additive itself, which may be used in inks used in the electro-optical display described above, a general structure for such molecular additives including a three-arm structure may be:

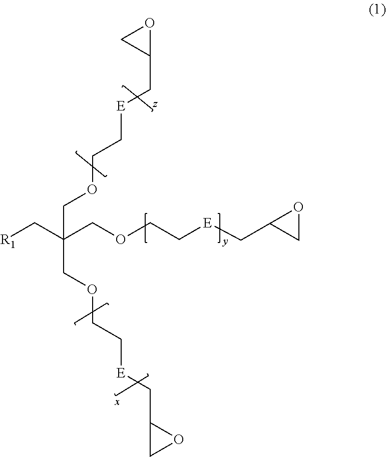

wherein R₁ is selected from the group consisting of hydrogen, saturated hydrocarbons, and unsaturated hydrocarbons, wherein if R₁ is a saturated or unsaturated hydrocarbon, such hydrocarbon may be substituted or non-substituted; wherein E is selected from the group consisting of CH₂, O, S, and NH, wherein “C” is carbon, “O” is oxygen, “S” is sulfur, “N” is nitrogen, and “H” is hydrogen; and wherein x, y, and z are each independently any whole number from 0 to 45, inclusive, wherein the sum of x, y, and z is less than 45 or equal to 45. In some examples, specific examples of R₁ may include hydrogen, alkyl groups, branched alkyl groups, aliphatic or aromatic acyl groups, alkenyl groups, or branched alkenyl groups. In examples wherein R₁ is substituted, examples of such substitution groups include, but are not limited to, hydrocarbons, such as alkyls, alkoxy groups or other like groups.

In another example, a general structure for the small molecular additive described herein including a two-arm structure may be:

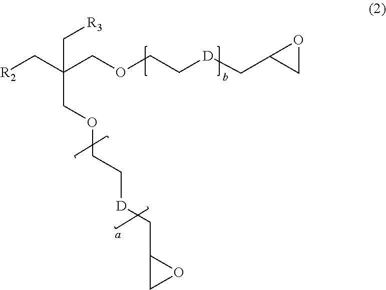

wherein R₂ and R₃ are each independently selected from the group consisting of hydrogen, saturated hydrocarbons, and unsaturated hydrocarbons, wherein if R₂ or R₃ is a saturated or unsaturated hydrocarbon, such hydrocarbon may be substituted or non-substituted; wherein D is selected from the group consisting of CH₂, O, S, and NH, wherein “C” is carbon, “O” is oxygen, “S” is sulfur, “N” is nitrogen, and “H” is hydrogen; and wherein a and b are each independently any whole number from 0 to 45, inclusive, wherein the sum of a and b is less than 45 or equal to 45. In some examples, R₂ and R₃ may each independently be selected from the group consisting of hydrogen, alkyl groups, branched alkyl groups, aliphatic or aromatic acyl groups, alkenyl groups, and branched alkenyl groups. In examples wherein R₂, R₃ or both R₂ and R₃ are substituted, examples of such substitution groups include, but are not limited to, hydrocarbons, such as alkyls, alkoxy groups or other like groups.

In one specific example, a general structure for the epoxide-based molecular additive, wherein x, y, and z in Structure (1) are all 0, may be:

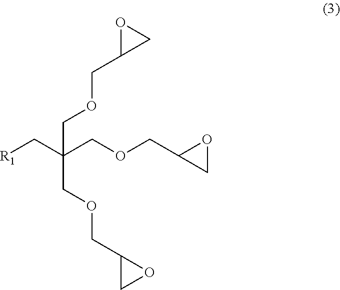

wherein R₁ is selected from the group consisting of hydrogen, saturated hydrocarbons, and unsaturated hydrocarbons, wherein if R₁ is a saturated or unsaturated hydrocarbon, such hydrocarbon may be substituted or non-substituted. Examples of non-substituted hydrocarbons may include hydrogen, alkyl groups, branched alkyl groups, aliphatic or aromatic acyl groups, alkenyl groups, or branched alkenyl groups. In examples, wherein R₁ is substituted, examples of such substitution groups may include, but are not limited to, any hydrocarbon, such as alkyls, alkoxy groups or other like groups.

As briefly discussed previously, when used in inks, the epoxide-based molecular additives may be added to the ink after being grafted onto the surface of a pigment particle by a covalent bond reaction between a functional group on the pigment particle and the epoxide group on the small molecular additive.

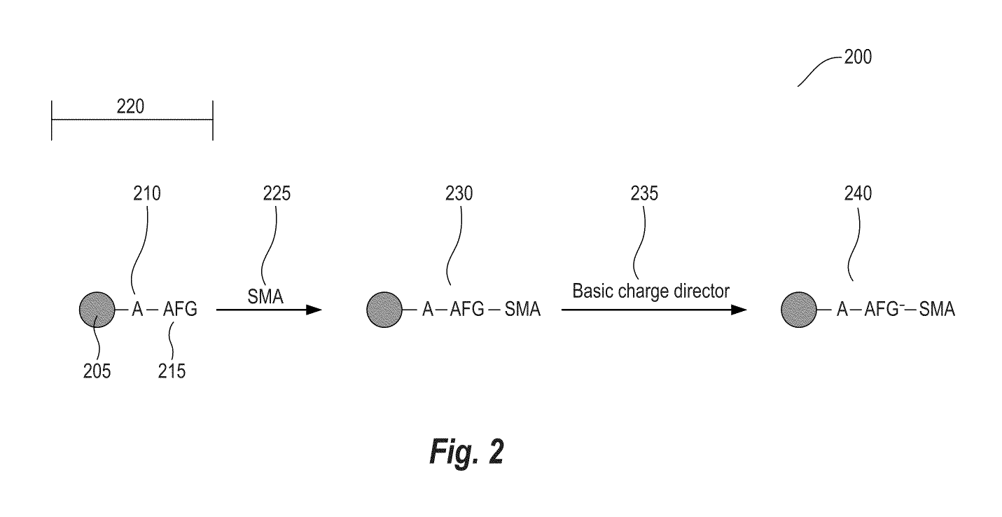

FIG. 2 is a schematic diagram 200 of an example reaction scheme for how an epoxide-based small molecular additive 225 may be grafted onto the surface of a pigment particle 230. In the schematic diagram 200, first, a surface modified pigment particle 220 may react with an epoxide-based small molecular additive as described above (and seen in FIG. 2 as “SMA”) 225 through a ring opening reaction, which may result in a pigment particle covalently bonded with the epoxide-based small molecular additive 230. In some examples, the surface modified pigment particle 220 may include a spacing group seen in FIG. 2 as “A” 210, an acidic functional group seen in FIG. 2 as “AFG” 215, and a pigment particle 205.

In some examples, the spacing group 210 may connect the pigment particle 205 with the acidic functional group 215 and may include any hydrocarbon or any aromatic ring. Specific examples of suitable spacing groups 210 may include, but are not limited to, any substituted aromatic ring, such as benzene derivatives, substituted benzene derivatives, naphthalene derivatives or substituted naphthalene derivatives; or hetero-aromatic derivatives such as pyridine derivatives, pyrimidine derivatives, triazine derivatives or furan derivatives.

In some examples, the acidic functional group 215 may be any acidic functional group. Specific examples of acidic functional groups 215 may include, but are not limited to, —OH, —SH, —COOH, —CSSH, —COSH, —SO₃H, —PO₃H, —OSO₃H, and —OPO₃H, wherein “O” is oxygen, “H” is hydrogen, “S” is sulfur, “C” is carbon, and “P” is phosphorus. Additionally, in some examples wherein a spacing group 210 is connected to an acidic functional group 215, only one acidic functional group 215 is connected to the spacing group 210. In other examples, two or more acidic functional groups 215 may be connected to a spacing group 210.

In some examples, the pigment particle, if added to an ink such as an electronic ink, may provide color and charge to the ink. Also, in response to a sufficient electric potential or field applied to the pigment particles while driving electrodes in the display, as described above, the pigment particles may move or rotate in the carrier fluid to different spots in the area of the display viewable by a user to produce different images. Different pigment particles may have different characteristics, such as different sizes, dispersibility properties, hues, colors or lightness. Additionally, different pigment particles may be further functionalized to contain different functional groups, which may further vary properties of the particle, including, but not limited to, hydrophilicity and hydrophobicity, acidity and basicity, or density of the particles.

The pigment particle may be a colored pigment or colored polymeric particle in any possible color, such as RGB or CYMK, with a size ranging from 10 nm to 10 μm. In some examples, smaller particles, with a particle size from 1 to 10 nm, such as quantum dots, may be employed. In other examples, the particle size may range to a few micrometers. Additionally, as further described below, the pigment particle may further include an inorganic coating layer such as silicon dioxide (SiO₂) or titanium dioxide (TiO₂), which may facilitate surface modification of the pigment particle. Finally, organic or inorganic pigments may be used.

Organic and inorganic pigment particles may be selected from black pigment particles, yellow pigment particles, magenta pigment particles, red pigment particles, violet pigment particles, cyan pigment particles, blue pigment particles, green pigment particles, orange pigment particles, brown pigment particles, white pigment particles or combinations thereof. In some instances, the organic or inorganic pigment particles may include spot-color pigment particles, which may be formed from a combination of a predefined ratio of two or more primary color pigment particles.

In some examples, non-limiting specific examples of inorganic black pigments may include carbon blacks such as No. 2300, No. 900, MCF88, No. 33, No. 40, No. 45, No. 52, MA7, MA8, MA100 or No. 2200B manufactured by Mitsubishi Chemical Corporation; Raven 5750, Raven 5250, Raven 5000, Raven 3500, Raven 1255 or Raven 700 manufactured by Columbian Chemicals Company; Regal 400R, Regal 330R, Regal 660R, Mogul L, Monarch 700, Monarch 800, Monarch 880, Monarch 900, Monarch 1000, Monarch 1100, Monarch 1300 or Monarch 1400 manufactured by Cabot Corporation; or Color Black FW1, Color Black FW2, Color Black FW2V, Color Black FW18, Color Black FW200, Color Black S150, Color Black S160, Color Black S170, Printex 35, Printex U, Printex V, Printex 140U, Special Black 6, Special Black 5, Special Black 4A or Special Black 4 manufactured by Degussa Corporation. In other examples, specific examples of organic black pigments may include aniline black (C.I. Pigment Black 1).

In other examples, non-limiting examples of suitable yellow organic pigments may include C.I. Pigment Yellow 1, C.I. Pigment Yellow 2, C.I. Pigment Yellow 3, C.I. Pigment Yellow 4, C.I. Pigment Yellow 5, C.I. Pigment Yellow 6, C.I. Pigment Yellow 7, C.I. Pigment Yellow 10, C.I. Pigment Yellow 11, C.I. Pigment Yellow 12, C.I. Pigment Yellow 13, C.I. Pigment Yellow 14, C.I. Pigment Yellow 16, C.I. Pigment Yellow 17, C.I. Pigment Yellow 24, C.I. Pigment Yellow 34, C.I. Pigment Yellow 35, C.I. Pigment Yellow 37, C.I. Pigment Yellow 53, C.I. Pigment Yellow 55, C.I. Pigment Yellow 65, C.I. Pigment Yellow 73, C.I. Pigment Yellow 74, C.I. Pigment Yellow 75, C.I. Pigment Yellow 81, C.I. Pigment Yellow 83, C.I. Pigment Yellow 93, C.I. Pigment Yellow 94, C.I. Pigment Yellow 95, C.I. Pigment Yellow 97, C.I. Pigment Yellow 98, C.I. Pigment Yellow 99, C.I. Pigment Yellow 108, C.I. Pigment Yellow 109, C.I. Pigment Yellow 110, C.I. Pigment Yellow 113, C.I. Pigment Yellow 114, C.I. Pigment Yellow 117, C.I. Pigment Yellow 120, C.I. Pigment Yellow 124, C.I. Pigment Yellow 128, C.I. Pigment Yellow 129, C.I. Pigment Yellow 133, 138, C.I. Pigment Yellow 139, C.I. Pigment Yellow 147, C.I. Pigment Yellow 151, C.I. Pigment Yellow 153, C.I. Pigment Yellow 154, C.I. Pigment Yellow 167, C.I. Pigment Yellow 172 or C.I. Pigment Yellow 180.

Non-limiting examples of suitable magenta, red or violet organic pigments may include C.I. Pigment Red 1, C.I. Pigment Red 2, C.I. Pigment Red 3, C.I. Pigment Red 4, C.I. Pigment Red 5, C.I. Pigment Red 6, C.I. Pigment Red 7, C.I. Pigment Red 8, C.I. Pigment Red 9, C.I. Pigment Red 10, C.I. Pigment Red 11, C.I. Pigment Red 12, C.I. Pigment Red 14, C.I. Pigment Red 15, C.I. Pigment Red 16, C.I. Pigment Red 17, C.I. Pigment Red 18, C.I.

Pigment Red 19, C.I. Pigment Red 21, C.I. Pigment Red 22, C.I. Pigment Red 23, C.I. Pigment Red 30, C.I. Pigment Red 31, C.I. Pigment Red 32, C.I. Pigment Red 37, C.I. Pigment Red 38, C.I. Pigment Red 40, C.I. Pigment Red 41, C.I. Pigment Red 42, C.I. Pigment Red 48(Ca), C.I. Pigment Red 48(Mn), C.I. Pigment Red 57(Ca), C.I. Pigment Red 57:1, C.I. Pigment Red 88, C.I. Pigment Red 112, C.I. Pigment Red 114, C.I. Pigment Red 122, C.I. Pigment Red 123, C.I. Pigment Red 144, C.I. Pigment Red 146, C.I. Pigment Red 149, C.I. Pigment Red 150, C.I. Pigment Red 166, C.I. Pigment Red 168, C.I. Pigment Red 170, C.I. Pigment Red 171, C.I. Pigment Red 175, C.I. Pigment Red 176, C.I. Pigment Red 177, C.I. Pigment Red 178, C.I. Pigment Red 179, C.I. Pigment Red 184, C.I. Pigment Red 185, C.I. Pigment Red 187, C.I. Pigment Red 202, C.I. Pigment Red 209, C.I. Pigment Red 219, C.I. Pigment Red 224, C.I. Pigment Red 245, C.I. Pigment Violet 19, C.I. Pigment Violet 23, C.I. Pigment Violet 32, C.I. Pigment Violet 33, C.I. Pigment Violet 36, C.I. Pigment Violet 38, C.I. Pigment Violet 43 or C.I. Pigment Violet 50.

Non-limiting examples of blue or cyan organic pigments may include C.I. Pigment Blue 1, C.I. Pigment Blue 2, C.I. Pigment Blue 3, C.I. Pigment Blue 15, C.I. Pigment Blue 15:3, C.I. Pigment Blue 15:34, C.I. Pigment Blue 15:4, C.I. Pigment Blue 16, C.I. Pigment Blue 18, C.I. Pigment Blue 22, C.I. Pigment Blue 25, C.I. Pigment Blue 60, C.I. Pigment Blue 65, C.I. Pigment Blue 66, C.I. Vat Blue 4 or C.I. Vat Blue 60.

Other non-limiting examples of green, brown, or orange organic pigments may include C.I. Pigment Green 7, C.I. Pigment Green 10, C.I. Pigment Brown 3, C.I. Pigment Brown 5, C.I. Pigment Brown 25, C.I. Pigment Brown 26, C.I. Pigment Orange 1, C.I. Pigment Orange 2, C.I. Pigment Orange 5, C.I. Pigment Orange 7, C.I. Pigment Orange 13, C.I. Pigment Orange 14, C.I. Pigment Orange 15, C.I. Pigment Orange 16, C.I. Pigment Orange 24, C.I. Pigment Orange 34, C.I. Pigment Orange 36, C.I. Pigment Orange 38, C.I. Pigment Orange 40, C.I. Pigment Orange 43 or C.I. Pigment Orange 63.

In the second step of the reaction depicted in FIG. 2, the pigment particle covalently bonded with the epoxide-based molecular additive 230 may be charged by a charge director 235 through an acid-base reaction, which may result in a more stable and charged pigment compound 240. In one example, the charge director may be basic and may react with the functionalized pigment particle to negatively charge the particle. In other words, the charging of the particle may be accomplished via an acid-base reaction (or interaction) between the charge director and the acid-modified particle surface. In examples wherein, such pigments are used in electronic inks, the charge director may also be used in the ink to prevent undesirable aggregation of the pigment particles in the ink. In other examples, the charge director may be acidic and may react (or interact) with the base-modified pigment particle to positively charge the particle. Again, the charging of the particle may be accomplished via an acid-base reaction (or interaction) between the charge director and the base-modified particle surface.

The charge director may be selected from small molecules or polymers that may be capable of forming reverse micelles in a non-polar carrier fluid. Such charge directors may be colorless and may be dispersible or soluble in the carrier fluid. As discussed above, examples of charge directors include, but are not limited to, neutral and non-dissociable charge directors such as polyisobutylene succinimide amines; Chevron Corporation's Oronite dispersant; ionizable charge directors that may disassociate to form charges such as sodium di-2-ethylhexylsulfosuccinate dioctyl sulfosuccinate (AOT); zwitterionic charge directors such as Lecithin; and non-chargeable and neutral charge directors, which may not disassociate or react with acids or bases to form charges, such as fluorosurfactants.

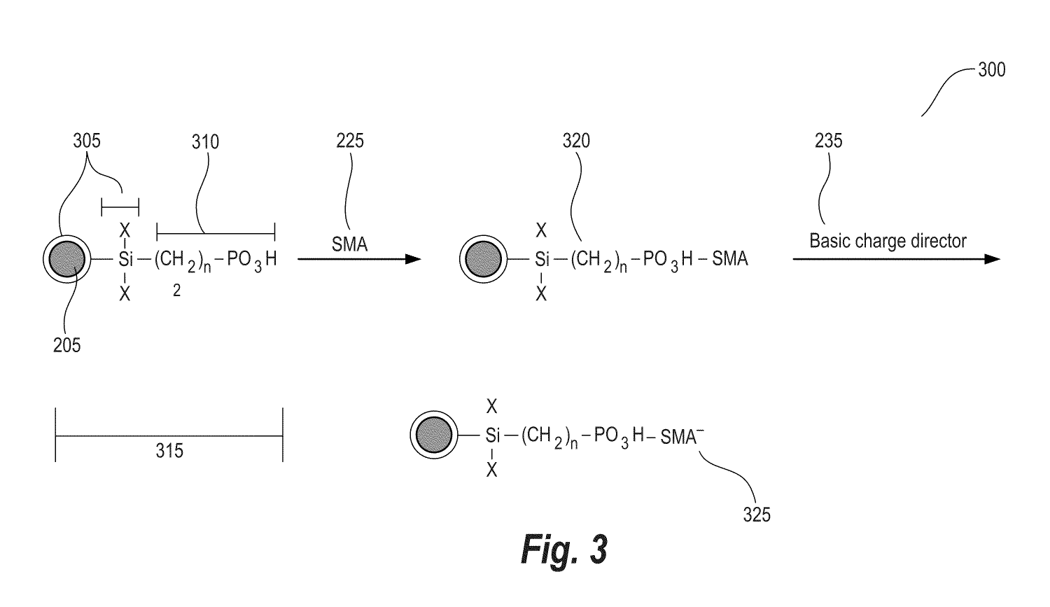

FIG. 3 is a schematic diagram 300 of a specific example reaction scheme for how an epoxide-based small molecular additive 225 may be grafted onto the surface of a phosphoric acid surface modified, silica coated pigment particle 315. In this example, instead of an acidic functional group, as seen above in FIG. 1, a nucleophile 310, such as phosphoric acid, may be used to bond the epoxide-based molecular additive. In other examples, any nucleophile may be used. Specific examples of suitable nucleophiles may include, but are not limited to, —OH, —SH or —NH groups. Additionally, in this example, the nucleophile 310 may be combined with the pigment particle 205 through surface modification. Such surface modification may be facilitated through use of an inorganic coating 305 on the pigment particle, as further described above. In one specific example, the inorganic coating 305 in FIG. 2 is a silicon-based coating, wherein X may be oxygen, any halogen, such as chlorine, bromine or iodine, or any alkyloxy group, such as methoxy, ethoxy or propoxy. The nucleophile in FIG. 2 is the phosphoric acid group 310, wherein n is any integer between 0 and 18, inclusive.

In the schematic diagram 300, first, a surface modified pigment particle 315 may react with an epoxide-based small molecular additive 225 through a ring opening reaction, which may result in a pigment particle covalently bonded with the epoxide-based molecular additive 320. Second, the compound 320 is further charged and stabilized by using a charge director 235 in an acid-base interaction, as described above in FIG. 2.

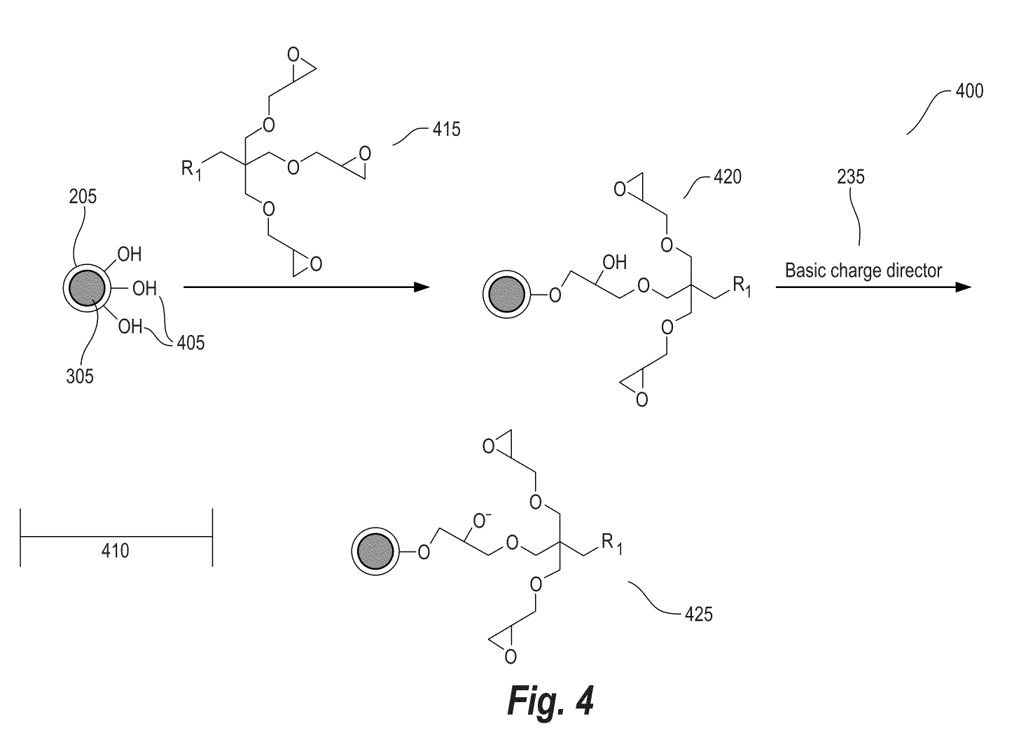

FIG. 4 is a schematic diagram 400 of a specific example reaction scheme for how an epoxide-based small molecular additive 415 may be grafted onto a hydroxyl group surface modified, silica coated pigment particle 410. In such an example, a hydroxyl group 405 acts as a nucleophile, serving a similar function as the phosphoric acid group described above in FIG. 3. Additionally, in this example, the pigment particle 205 is coated with an inorganic coating 305, as described above, wherein such coating may facilitate surface modification of the pigment particle 205.

In the schematic diagram 400, first, a surface modified pigment particle 410 may react with an epoxide-based small molecular additive 415 through a ring opening reaction, which may result in a pigment particle covalently bonded with the epoxide-based molecular additive 420. Second, the compound 420 is further charged and stabilized by using a charge director 235 in an acid-base interaction, as described above in FIG. 2.

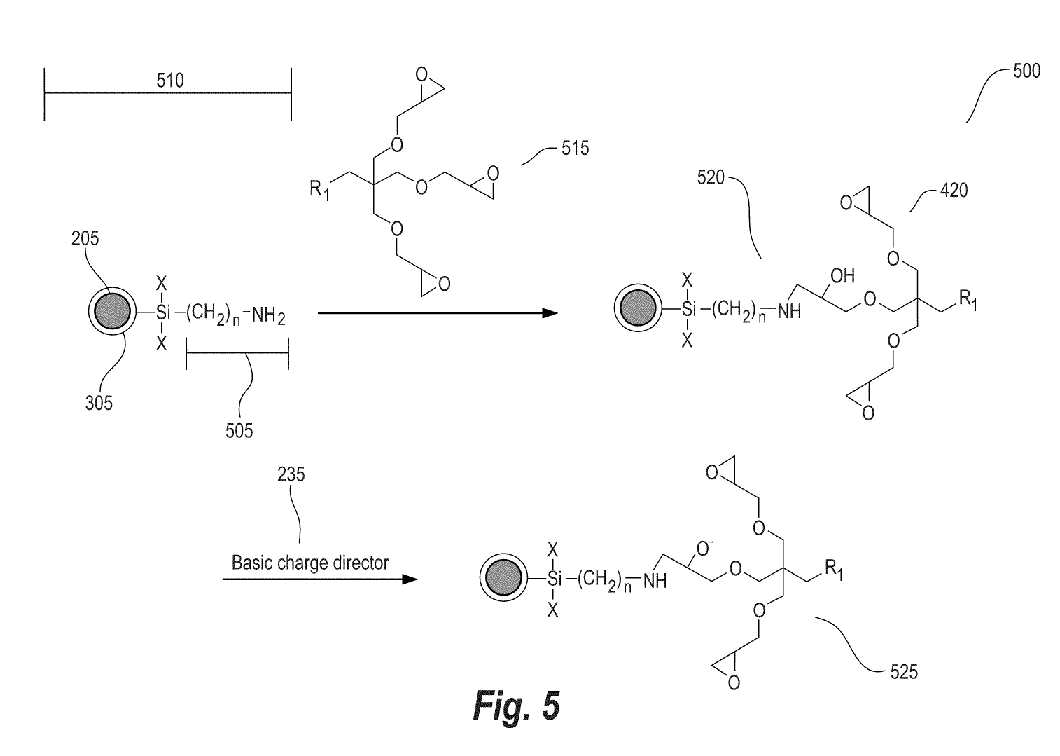

FIG. 5 is a schematic diagram 500 of a specific example reaction scheme for how an epoxide-based small molecular additive 515 may be grafted onto an amino group surface modified, silica coated pigment particle 510. In such an example, an amino group 505 acts as a nucleophile, serving a similar function as the phosphoric acid group described above in FIG. 3. Additionally, in this example, the pigment particle 205 is coated with an inorganic coating 305, which may facilitate surface modification of the pigment particle 205. In the example shown in FIG. 5, the inorganic coating is a silicon-based coating, wherein X may be oxygen, any halogen, such chlorine, bromine or iodine, or any alkyloxy group, such as methoxy, ethoxy or propoxy.

In the schematic diagram 500, first, a surface modified pigment particle 510 may react with an epoxide-based small molecular additive 515 through a ring opening reaction, which may result in a pigment particle covalently bonded with the epoxide-based molecular additive 520. Second, the compound 520 is further charged and stabilized by using a charge director 235 in an acid-base interaction, as described above in FIG. 2.

Turning now to inks that include the additive described herein and may be used in electro-optical displays described above in FIG. 1, such as electrokinetic/electrophoretic displays, examples of such electronic inks may generally include a non-polar carrier fluid and a functionalized pigment particle bonded to an epoxide-based small molecular additive as described herein. Additionally, in some examples, such electronic inks may further include other additives, such as other surfactants, dispersants or charge directors.

In some examples, the carrier fluid may act as a vehicle for dispersing the pigment particle as described herein and may act as an electrokinetic/electrophoretic medium. In one example, non-polar fluids are used, as such fluids may reduce leakages of electric current when driving the display and may increase the electric field present in the ink. In some examples, the non-polar carrier fluid may be a fluid having a low dielectric constant k such as, e.g., less than about 20 or, in some examples, less than about 2. In other examples, carrier fluids may also vary with respect to viscosity, resistivity, specific gravity, chemical stability or toxicity, wherein such differences may be considered when formulating an electronic ink. For example, a carrier fluid that is too viscous may slow down the spread or compaction of the pigment particles, which may affect switching speed and may result in a less effective electronic ink.

Specifically, in some examples, the non-polar carrier fluid may include one or more fluids selected from the group consisting of hydrocarbons, halogenated hydrocarbons, partially halogenated hydrocarbons, oxygenated fluids, siloxanes, and combinations thereof. Some specific examples of non-polar carrier fluids may include, but are not limited to, perchloroethylene, cyclohexane, dodecane, mineral oil, isoparaffinic fluids, cyclopentasiloxane, cyclohexasiloxane, cyclooctamethylsiloxane or combinations thereof.

Additionally, in some examples, the electronic ink may further include other additives such as dispersants, charge directors (as described above), optical brighteners, polymers, rheology modifiers, surfactants, viscosity modifiers or combinations thereof. Such additives may serve to modify properties of an ink including, but not limited to, viscosity or brightness.

In some examples, the concentration of pigment particles and other additives, such as dispersants, charge directors, or surfactants, in the ink, may range from about 0.5 to 20 percent by weight (wt %). In one example, the concentration of functionalized pigment particles bonded to a small epoxide-based molecular additives as described herein in the ink may range from about 1 to 10 wt %. The carrier fluid makes up the balance of the ink.

It should be understood that while the electronic inks including the epoxide-based molecular additives discussed above have been described with specific reference to electrophoretic/electrokinetic applications, such additives may find use in other applications as well, including, but not limited to, liquid electrophotographic printing applications.

What is claimed is:

1. A compound having a three-arm structure: 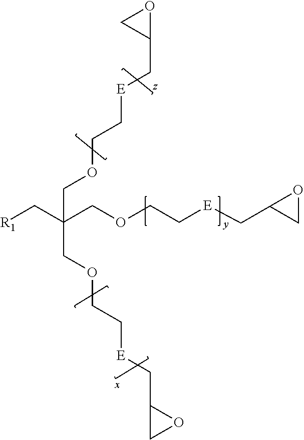
wherein R₁ is selected from the group consisting of hydrogen, substituted saturated hydrocarbons, non-substituted saturated hydrocarbons, substituted unsaturated hydrocarbons, and non-substituted unsaturated hydrocarbons; wherein E is selected from the group consisting of CH₂, O, S, and NH; and wherein x, y, and z are each independently any whole number between 0 and 45, inclusive, wherein the sum of x, y, and z is less than 45 or equal to 45; or having a two-arm structure: 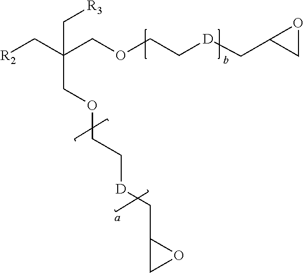
wherein R₂ and R₃ are each independently selected from the group consisting of hydrogen, substituted saturated hydrocarbons, non-substituted saturated hydrocarbons, substituted unsaturated hydrocarbons, and non-substituted unsaturated hydrocarbons; wherein D is selected from the group consisting of CH₂, O, S, and NH; and wherein a and b are each independently any whole number between 0 and 45, inclusive, wherein the sum of a and b is less than 45 or equal to 45.

2. The compound of claim 1 having the three-arm structure wherein x, y, and z are all 0.

3. The compound of claim 1 wherein R₁, R₂, and R₃ are each independently selected from the group consisting of alkyls, branched alkyls, aliphatic acyls, aromatic acyls, alkenyls, and branched alkenyls and wherein R₁, R₂, and R₃ are each independently substituted or non-substituted.

4. The compound of claim 1 having the three-arm structure, wherein R₁ is substituted with a group selected from the group consisting of alkoxy groups, alkyl groups, alkene groups, and alkyne groups; or having the two-arm structure, wherein R₂, R₃ or R₂ and R₃ are substituted with a group selected from the group consisting of alkoxy groups, alkyl groups, alkene groups, and alkyne groups.

5. An ink including:
a non-polar carrier fluid; and
a pigment particle covalently bonded to a compound, wherein the compound has a three-arm structure: 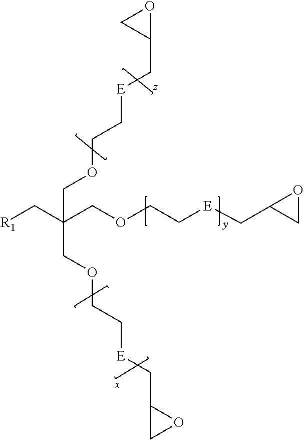
wherein R₁ is selected from the group consisting of hydrogen, substituted saturated hydrocarbons, non-substituted saturated hydrocarbons, substituted unsaturated hydrocarbons, and non-substituted unsaturated hydrocarbons; wherein E is selected from the group consisting of CH₂, O, S, and NH; and wherein x, y, and z are each independently any whole number between 0 and 45, inclusive, wherein the sum of x, y, and z is less than 45 or equal to 45; or wherein the compound has a two-arm structure: 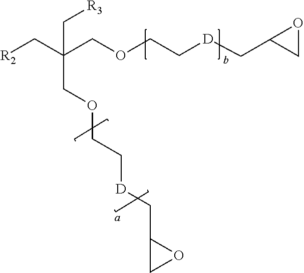
wherein R₂ and R₃ are each independently selected from the group consisting of hydrogen, substituted saturated hydrocarbons, non-substituted saturated hydrocarbons, substituted unsaturated hydrocarbons, and non-substituted unsaturated hydrocarbons; wherein D is selected from the group consisting of CH₂, O, S, and NH; and wherein a and b are each independently any whole number between 0 and 45, inclusive, wherein the sum of a and b is less than 45 or equal to 45.

6. The ink of claim 5 wherein the compound has the three-arm structure and wherein x, y, and z are all 0.

7. The ink of claim 5 wherein the compound has the three-arm structure and wherein R₁ is substituted with a group selected from the group consisting of alkoxy groups, alkyl groups, alkene groups, and alkyne groups; or wherein the compound has the two-arm structure and wherein R₂, R₃ or R₂ and R₃ are substituted with a group selected from the group consisting of alkoxy groups, alkyl groups, alkene groups, and alkyne groups.

8. The ink of claim 5 wherein the non-polar carrier fluid is a non-polar solvent selected from the group consisting of hydrocarbons, halogenated hydrocarbons, partially halogenated hydrocarbons, siloxanes, and combinations thereof.

9. The ink of claim 5 wherein the pigment particle is surface-modified to include a functional group.

10. The ink of claim 5 wherein the pigment particle further includes an inorganic coating.

11. The ink of claim 5 wherein the pigment particle is selected from the group consisting of black pigment particles, yellow pigment particles, magenta pigment particles, red pigment particles, violet pigment particles, cyan pigment particles, blue pigment particles, green pigment particles, orange pigment particles, brown pigment particles, white pigment particles, and combinations thereof.

12. The ink of claim 5 further including an additive selected from the group consisting of dispersants, charge directors, optical brighteners, polymers, rheology modifiers, surfactants, viscosity modifiers, and combinations thereof, and wherein if the additive is a charge director, the charge director is a small molecule or polymer that is capable of forming reverse micelles in the non-polar carrier fluid.

13. In combination, an electronic display and an ink, wherein the electronic display includes:
a first electrode;
a second electrode; and
a display cell having a recess defined by a dielectric material, the first electrode, and the second electrode; wherein the display cell contains the ink; and wherein the ink includes:
a non-polar carrier fluid; and
a pigment particle covalently bonded to a compound, wherein the compound has a three-arm structure: 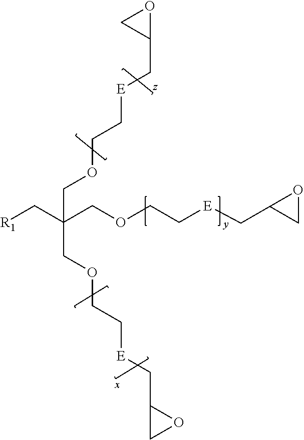
wherein R₁ is selected from the group consisting of hydrogen, substituted saturated hydrocarbons, non-substituted saturated hydrocarbons, substituted unsaturated hydrocarbons, and non-substituted unsaturated hydrocarbons; wherein E is selected from the group consisting of CH₂, O, S, and NH; and
wherein x, y, and z are each independently any whole number between 0 and 45, inclusive, wherein the sum of x, y, and z is less than 45 or equal to 45; or wherein the compound has a two arm structure: 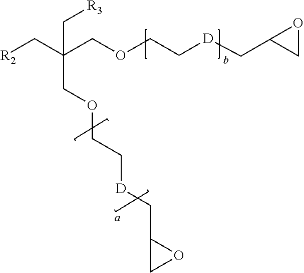
wherein R₂ and R₃ are each independently selected from the group consisting of hydrogen, substituted saturated hydrocarbons, non-substituted saturated hydrocarbons, substituted unsaturated hydrocarbons, and non-substituted unsaturated hydrocarbons; wherein D is selected from the group consisting of CH₂, O, S, and NH; and wherein a and b are each independently any whole number between 0 and 45, inclusive, wherein the sum of a and b is less than 45 or equal to 45.

14. The combination of claim 13 wherein the electronic display includes a plurality of display cells in a stacked configuration, associated first electrodes and second electrodes, and a plurality of inks of different colors, each display cell containing an ink of a different color.

15. The combination of claim 13 wherein the compound has the three-arm structure and wherein R₁ is substituted with a group selected from the group consisting of alkoxy groups, alkyl groups, alkene groups, and alkyne groups; or wherein the compound has the two-arm structure and wherein R₂, R₃ or R₂ and R₃ are substituted with a group selected from the group consisting of alkoxy groups, alkyl groups, alkene groups, and alkyne groups.

16. The combination of claim 13 wherein the non-polar carrier fluid is a non-polar solvent selected from the group consisting of hydrocarbons, halogenated hydrocarbons, partially halogenated hydrocarbons, siloxanes, and combinations thereof.

17. The combination of claim 13 wherein the pigment particle is surface-modified to include a functional group.

18. The combination of claim 13 wherein the pigment particle further includes an inorganic coating.

19. The combination of claim 13 wherein the pigment particle is selected from the group consisting of black pigment particles, yellow pigment particles, magenta pigment particles, red pigment particles, violet pigment particles, cyan pigment particles, blue pigment particles, green pigment particles, orange pigment particles, brown pigment particles, white pigment particles, and combinations thereof.

20. The combination of claim 13, wherein the ink further includes an additive selected from the group consisting of dispersants, charge directors, optical brighteners, polymers, rheology modifiers, surfactants, viscosity modifiers and combinations thereof, and wherein if the additive is a charge director, the charge director is a small molecule or polymer that is capable of forming reverse micelles in the non-polar carrier fluid.
